(19) United States
(12) Patent Application Publication
(10) Pub. No.: US 2006/0166377 A1
(43) Pub. Date: Jul. 27, 2006

(54) Particle for magnetically induced membrane transport

(76) Inventors: Sarah Fredriksson, STAFFANSTORP (SE); Dario Kriz, Hoor (SE)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/526,901

(22) PCT Filed: Sep. 11, 2003
(86) PCT No.: PCT/SE03/01412

§ 371 Date: Apr. 6, 2005

(30) Foreign Application Priority Data
Sep. 12, 2002 (SE) 0202725-8

(51) Int. Cl. G01N 33/553

(52) U.S. Cl. 436/526

(57) ABSTRACT

The present invention relates to particles, for use in transport of substances through biological membranes, the particle being characterised in that it contains at least one magnetically inducible material and at least one difunctional molecule with at least one binding site for said substance and at least one binding site for said biological membrane. Moreover the invention concerns both production of the same particles and applications thereof.

### FIELD OF THE INVENTION

The present invention relates to a particle containing magnetically inducible material intended for transport of substances through biological membranes.

### BACKGROUND ART

A biological cell, whether it is a human cell, a bacterium or some other type of cell, is enclosed by a cell membrane. This membrane is often made up of a double layer of phospholipids. The more hydrophobic parts of the lipids form the interior of the membrane while the hydrophilic part are oriented towards the interior of the cell and towards the surroundings. Furthermore the cell membranes contain many different proteins. Different types of proteins in the membrane have different practical tasks of importance to the life cycle of the cell. Some proteins serve as transport channels for different types of ions and small metabolites. Other proteins, receptors, give the membrane properties causing different biochemical signals from the surroundings to be registered by the cell. The membrane protects the cell from the surroundings and performs a selective control of the flow of molecules to and from the cell.

To force a certain recipient cell to receive a molecule, which is usually not admitted by the cell, research scientists are using various methods. Molecules that are not too large can be modified and masked so as to resemble a molecule which is automatically admitted by the cell. For larger molecules such as proteins and DNA molecules, use is made of either physical methods which open the cell membrane, or virus particles. A virus may carry relatively large DNA molecules. The virus recognises a certain type of cells and can introduce the DNA molecule which it carries, through the cell membrane. Since viruses themselves are a potential danger to laboratory staff, new virus-like particles have been made of liposomes to try to imitate the virus capability of introducing DNA. Moreover, existing viruses have been modified to reduce the risk of handling in laboratory environment. Physical treatment of cells, such as electric shock, heat shock or using gene bombardment or gene cannon, destroys or widens the cell membrane temporarily and the membrane will be temporarily open for influx of a larger molecule. The physical methods are not specific and involve all cells in a sample. It is also common for these methods to kill a large number of cells. To introduce DNA or protein material into a single cell with great precision, microinjection under a microscope may be an alternative, but only one cell at a time can be treated. The development of new technologies for introduction of DNA into cells has accelerated during the last decade with increasing knowledge of our genes. The development of so-called stem cells and other cell lines, where the cells do not divide very often or not at all, has increased the need for methods which can introduce DNA not only through a cell membrane but also through the membrane of the nucleus so that the DNA molecule reaches all the way to the nucleus.

A method for affecting cell membranes has previously been described, see Fredriksson S. and Kriz D. WO01/18168 “Device for introducing pores into biological materials”. The method referred to as magnetoporation uses ferromagnetic particles. These particles have a diameter of 1-100 nm. By modification of their surface, the particles can be made to bind to a cell membrane. Then the cell and the particle complexes are exposed to an alternating magnetic field. The ferromagnetic particles then emit heat and vibrate slightly. The cell membrane will be more permeable in the vicinity of the particle and, molecules, such as DNA, can diffuse through the membrane. Alternatively, the entire particle can penetrate the membrane.

The present invention describes the above-mentioned ferromagnetic particle as a particle for transporting a molecule through one or more membranes without this particle necessarily being transported through the membrane.

Magnetically inducible particles, in the size of ten nanometres up to a few micrometres, with modified surfaces are commercially available for various purposes, such as contrast medium for MRI (magnetic resonance imaging), preparation of RNA (ribonucleic acid) and DNA (deoxyribonucleic acid), synthesis of cDNA library on solid phase, protein purification, carrier in immunological analyses, markers in immunological analyses, ion exchange and affinity chromatography and purification or sorting of cells, viruses and organelles.

The main component of commercially available magnetically inducible particles is in most cases ferrite/magnetite, a special type of iron oxide having magnetic properties, i.e. its relative magnetic permeability is very high. The particles contain a core of iron oxide and/or iron oxide hydroxide and sometimes one more or several metals and its/their oxides. These paramagnetic cores are not permanent magnets in themselves, but if the magnetic domains in the core of each particle are exposed to a magnetic field, they try to adjust to the direction of the field. As the effect of the field decreases, the magnetic domains gradually lose their direction. If the paramagnetic particles are exposed to a magnetic field that changes direction at a frequency in the order of 1 MHz, the particles will in each change of direction of the field carry an initial counterforce opposite to the direction of the field before the magnetic domains change direction. This phenomenon which has been schematically explained above can be derived from the hysteresis curve for ferromagnetic materials and results in loss of energy which is notable in the form of heat development from the particles.

Each core may consist of a magnetic domain or a plurality of domains which via aggregation have formed a somewhat greater complex. Most of the cores of the commercially available particles consist of more than one magnetic domain. The size of the core of the particle determines whether it is possible to quickly and easily separate the magnetic particles from a heterogeneous mixture with a magnetic field (frequently a simple permanent magnet). For all applications of magnetic particles involving purification or sorting of a specific component from a large number of other components, this is very practical, and these particles often have a diameter greater than 200 nm. For the particle according to the present invention, however, it is important for it to be so small that it does not settle by gravity and does not aggregate with neighbouring particles and form a greater complex when storing said particle in a water-based suspension. It is also most important that the particle according to the invention can be handled without inducing infection of some kind in a target cell. The particle must therefore without difficulty pass a sterile filter with the size of 100-200 nm. The particles according to the present invention thus form a stable ferrofluid, cf. Massart et al U.S. Pat. No. 4,329,241, i.e. a stable colloidal suspension of ferromagnetic particles. This means that the particles stay in the suspension and that by diffusion they can move in a cell suspension and find their target.

The core of commercially available particles is often enclosed in or mixed with a polymer, such as dextran or protein, or enclosed by an outer monolayer or bilayer of amphiphatic molecules, such as fatty acids or derivatives thereof. This outer envelope counteracts aggregation of neighbouring cores that may otherwise occur. The outer envelope also facilitates extension of the particle and has been used for chemical bonding of other molecules, for instance receptors, lectins, enzymes and antibodies, to the surface of the magnetically inducible particles, whereby they obtain selectively binding properties. The binding properties make the particle bond to a target object. It is of great importance to the particle according to the invention that the particles do not aggregate since the size of each particle must be in the order of about 1 to about 200 nm for the particles to be kept stable in a suspension and not to settle and also be simple to sterile-filter in the case where this is desirable. An outer envelope which counteracts aggregation is thus necessary. At the same time it is important that the heat emitted from the core of the particle reach the surroundings. In the present invention, this conflict has been solved by means of a particle made up of a core which is not mixed or enclosed by polymer and is also not coated with a mono- or bilayer of amphiphatic molecules, but a core produced in a water-based system, cf. Massart et al U.S. Pat. No. 4,329,241. The core is stabilised in two different ways depending on what type of molecule is to be bonded to the particle and there constitute the selectively binding and effector carrying part of the particle (see below). Either the core is stabilised by this molecule directly via van der Waals bonds to the metal oxide/hydroxide core or by a smaller molecule exemplified by an organic silanised molecule, succinic acid and its derivative or an amino acid. Then the selectively binding and effector carrying molecule is bonded covalently to this smaller molecule.

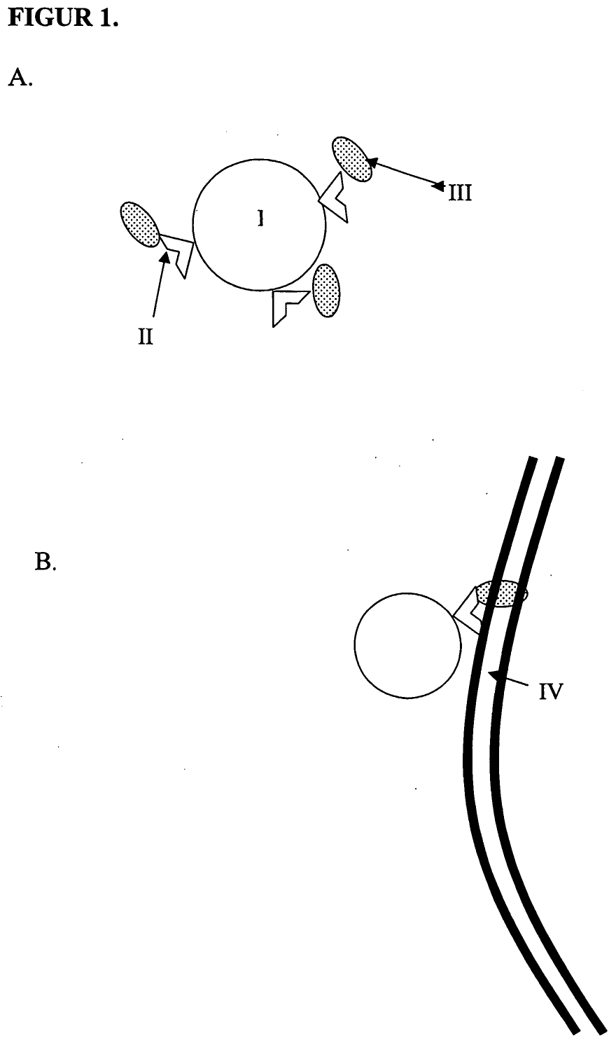

A further requirement placed on the particle described in the present invention is that it should be able to bring along a molecule to its target. This molecule can essentially be any molecule, but is exemplified by DNA, RNA and proteins and is here referred to as effector molecule. Furthermore, this effector molecule, unit III in FIG. 1A, must be located in the vicinity of the selectively binding molecule, unit II in FIG. 1A, on the core of said particle, unit I in FIG. 1A. Therefore, the selectively binding molecule and the effector carrying molecule must either be one and the same molecule, that is to say exhibit both properties, or two units are bonded together chemically or by genetic fusion to a single molecule. The purpose of this is to place said effector molecule adjacent to the point on the membrane, unit IV in FIG. 1B, where the selective molecule has bonded, see FIG. 1B. This point on the membrane is the only given point where the membrane will react to the heat and vibrations from the particle when this, in turn, is exposed to an alternating magnetic field. The temporary pore which then forms in the membrane and the increasing local diffusion owing to the development of heat cause said effector molecule to diffuse through the membrane even if it is not the entire particle that is entrained through the membrane. This effect is crucial when one does not want to introduce the entire particle through a membrane, for instance, in order not to disturb a living cell or nucleus more than necessary. This construction further makes the particle unique among previously described paramagnetic particles, cf. U.S. Pat. No. 4,329,241, U.S. Pat. No. 5,928,958, U.S. Pat. No. 6,150,181, PCT/EP00/09004, PCT/US01/03738 and PCT/US97/12657. By selecting how the effector molecule is bonded to the particle and selecting frequency, field strength, length of the pulse and number of pulses from the alternating field, the transport through the membrane with or without the entire particle can be regulated. Bonding with greater strength exemplified by affinity bonding is more likely to lead to transport of the entire particle through the membrane whereas electrostatic bonding or van der Waals bonding increases the possibility of regulating the membrane transport so that only the effector molecule is transported through the membrane.

It is previously known, U.S. Pat. No. 5,928,958, how a superparamagnetic particle of the size of 3 to 1000 nm consisting of a core of iron oxide enclosed by an organic molecule, to which many different molecules can be bonded, can be produced for various purposes. U.S. Pat. No. 5,928,958 also discloses how this type of particles can be used as tumour destructive agents, to increase an immune response for a molecule bonded to its surface, to direct, by means of a permanent magnet or electromagnetic field, a certain drug substance to a target organ, for purification of fused cells, for purification of cells having absorbed gene material bonded to the particle, as contrast medium, for in vitro diagnostics and as magnetic carriers or adsorbents. Independently of use and independently of how many molecules are bound to the surface of this particle, it is not described how these molecules are oriented in relation to each other, which makes the particle according to the invention unique. Furthermore, U.S. Pat. No. 5,928,958 does not describe how the particle is to be designed to be used for membrane transport in an alternating magnetic field.

Furthermore a particle of nanosize has previously been described, coated with a derivative of succinic acid bound in a second step to annexin designed for marking of molecules or cells with said magnet particles. In the present invention, a particle is described, on whose surface there are at least two unique properties with specific relative positions on the particle, which is unique. Bahr et al, PCT/EP00/09004 discloses a particle made up of an iron oxide core enclosed by a biocompatible substrate, to which different effector molecules can be bonded to which in turn biomolecules are bonded by covalent bonds. The particle according to the present invention has a minimum layer of molecules outside the core for its specific use and therefore differs materially from the particle according to Bahr et al.

It is previously known that particles of nanosize can be used to kill tumour cells by heating in an alternating electromagnetic field, referred to as hyperthermia. The particles for this purpose are modified with a stabilising envelope and a recognition molecule for a specific target cell. The cell particle complex is placed in an alternating field until the heat of the attacked target cells becomes so high that the cell dies. This application aims at completely knocking out a cell using heat. In the present invention, a particle is described, which can utilise the heat from the magnetically inducible core for a completely different purpose, viz. membrane transport, without affecting the entire cell with a general increase in temperature.

In the cases where the target of the effector molecule is an organelle inside a cell, passage through more than one membrane is required. A variant of the described particle is that it is enclosed by a lipid envelope which forms a liposome. In a first step, this magnetoliposome is allowed to fuse with the cell membrane. In the next step, the particle without the liposome envelope inside the cell is allowed to seek out the target membrane exemplified by the membrane of the nucleus, after which the cell is exposed to an alternating magnetic field once or repeatedly. This design of the particle is particularly important when introducing DNA into the nucleus in living cells which do not divide.

Magnetoliposomes for target-specific treatment of biological material have been described in the patent literature, see PCT/EP00/09004. The magnetic part of these liposomes is used to direct, by means of a magnetic field, the liposome to the correct target object, which differs from the present invention where the liposome transports the active magnetic particle through an outer cell membrane, after which the magnetic particle is used for one more membrane transport through an alternating magnetic field. Magnetofluorescent liposomes are disclosed in PCT/US97/12657 for specific marking of cells in cell sorting, which is not relevant to the present invention.

### SUMMARY OF THE INVENTION

The invention relates to a particle intended for magnetic field induced membrane transport of substances. The particle contains on the one hand a magnetically inducible component and, on the other, a membrane-binding component which at the same time also constitutes a substance-binding component. Preferably, the diameter of the particles is greater than about 1 nm and smaller than about 1 micrometre.

In one embodiment of the particle, said magnetically inducible component contains at least one metal or a derivative thereof, such as an oxide.

In another embodiment of the particle, the membrane transport effect of said particle can be induced by an applied alternating magnetic field with a vibration frequency in the range of about 10 Hz to about 100 MHz and a field strength in the range of about 1 to about 1000 Oerstedt.

In alternative embodiments of the particle, it also contains indicator materials and/or a bilayer membrane component which forms a liposome structure.

The particle according to the present invention can be used for biochemical work in analysis, preparation and research in laboratories. The effect of the particle can be additionally reinforced by a method where a suspension of the particle is first mixed with membrane-enclosed structures and is allowed to incubate for about 1 min to about 3 h before the formed particle membrane complex is exposed to an alternating magnetic field.

### DETAILED DESCRIPTION OF THE INVENTION

The particle according to the present invention is characterised by two components. One is a magnetically inducible core and the other is a molecule with two properties in one and same molecule, i.e. a difunctional molecule. The properties by which said molecule is defined are its capability of specifically recognising a target object and bonding to this and its capability of attracting an effector molecule so that this effector molecule is transported with the particle according to the invention.

The magnetically inducible core may consist of iron oxides or iron oxide hydroxides or mixtures thereof, and may contain oxides of other materials such as Co, Ni, Mn, Be, Mg, Ca, Ba, Sr, Cu, Zn, Pt, Al, Cr, Bi, rare earth metals or mixtures thereof. The core has a size of between about 1 and about 100 nanometres and in total the particle has a diameter of between 1 nanometre and about 1 micrometre.

The difunctional molecule can be a protein, a peptide, a hormone, an organic molecule, a DNA or RNA molecule which has a natural and strong affinity for a target object. This difunctional molecule can be exemplified by a lectin and its affinity for carbohydrates on the proteins of cell membranes or an antibody and its affinity for a certain antigen. These protein molecules often contain sufficient charges to be able to bind a molecule by electrostatic bonds or hydrophobic parts which can bind to other molecules by van der Waals interactions. As a rule, it is not this capability for which the molecule is known and therefore it is in many cases not documented. We have discovered, for instance, that the lectin Concanavalin A and rabbit IgG molecules bind plasmid-DNA enough to be able to transport it to a carbohydrate-containing cell membrane where it is bonded to a magnetically inducible particle, see example IV below. If the divalent function is not available in a molecule, it can be provided by combinations of either covalent binding or by gene fusion between at least two different molecules or parts thereof. A tetralysin peptide fused to a lectin gives the lectin a DNA associating site, see Example III below.

For the application of the particle according to the present invention in membrane transport, it is important to be able to follow and document the particle's path and location in or outside a cell. In an embodiment of the particle, a marker, such as a colourant, fluorescent material, radioactive material, chemoluminescent material or enzyme, is therefore bonded to the magnetically inducible core. In example II below, it is described how the enzyme luciferase is used for documentation of a variant of the particle and its capability of bonding to the outer cell membrane of E. coli bacteria.

In another design of the particle, it is enclosed by a phospholipid layer, which forms a liposome round the magnetically inducible core and the difunctional molecule bonded thereto. In this way, the particle can reach an organelle within a living cell and transport the effector molecule through an organelle membrane exemplified by the membrane, mitochondrial membrane or chloroplast membrane of the nucleus.

The production of the particle and examples of the application of the particle in membrane transport are exemplified below in more detail by the following, nonlimiting examples.

### EXAMPLE I

### Production Process for Particles with ConA as Difunctional Molecule on the Surface

A water-based slurry of aggregated iron oxide cores was prepared according to the method described by Massart, U.S. Pat. No. 4,329,241. Then the slurry was treated with distilled water, pH 3.0 adjusted with HCL (detergent solution) during sonication. After that the slurry was centrifuged (500 g, 10 min), and the excess solution was drained off. The pellets were then resuspended in the detergent solution and sonication followed by centrifuging was repeated until the particles did no longer settle. Then the g number of the centrifuging step was increased gradually in steps until the particle suspension was stable without settling during centrifuging for at least 1 h at 22,000 g. The particles were sterile-filtered. The suspension (1%, 10 mg/ml) exhibited a magnetic permeability of μᵣ=1.9. The iron content was measured to 49% by means of atomic adsorption. This suspension is called FF1. 0.1 ml FF1 was diluted 10 times in detergent solution. A solution of concanavalin A of 75 μg/ml in detergent solution was filtered through a desalting column (Pharmacia), whereupon 0.5 ml diluted FF1 and 0.5 ml concanavalin A solution were mixed in a test tube and allowed to be incubated for 30 min at room temperature on a rocking table. 1 ml bovine serum albumin solution (treated like concanavalin A above) of 250 μg/ml was added to the sample to coat the entire particle surface with protein, and this was incubated for 30 min at room temperature.

NaCl was added to the samples to a final concentration of 0.5 M to ensure that the particles were fully coated with protein, and to force the van der Waals interactions between the iron oxide particles and the protein molecules. The final sample was gel-filtered in PMS buffer in order to remove excess BSA molecules and exchange the buffer. Finally, the ferrofluid was sterile-filtered (0.2 μm).

### EXAMPLE II

### Visualisation of the Particles by Marking with Luciferase

The particles were produced as described above, but in this case the concanavalin A solution was mixed with luciferase (firefly) of 50 μg/ml. The ready-mixed luciferase/concanavalin A-ferrofluid was diluted until it exhibited μᵣ=1.0020. 20 μl of diluted ferrofluid as stated above was allowed to be incubated with 10 μl E. coli slurry (OD600=0.4, concentrated 10 times in PBS buffer) in 370 μl PBS buffer supplemented with 1 mM CaCl₂ and 1 mM MnCl₂ (PBS2). The cell ferrofluid suspension was allowed to be incubated for 30 min, after which the cells were centrifuged at 3000 g for 5 min and washed twice in PBS2. The cell particle pellets were resuspended in 50 μl beetle luciferin substrate from PROMEGA Luciferase assay system. The light intensity of luciferase confirmed bonding to the cells on the one hand in suspension and, on the other hand, in studies of the cells under a microscope.

### EXAMPLE III

### Transfection of E. coli LB121 with Plasmid-DNA pUC18 Bonded to ConA-Tetralysine

20 μl of a suspension according to the present invention where the cell-binding component consists of conA, which by gene fusion is expressed as a protein (expressed in E. coli) with the substance-binding component which consists of a synthetic polylysine peptide (4 amino acids), and where the concentration of the magnetically inducible component gives a suspension with a magnetic relative permeability of 1.002, was added to 0.5 μg pUC18 plasmid-DNA in 10 μl PBS. The sample was incubated for 20 min. 10 μl cells was grown to a cell density of OD (600 nm)=0.4 concentrated 10 times and resuspended 0.1 M PBS buffer containing 0.15 M NaCl was added to the ferrofluid. After incubation for 30 min, the sample was exposed for 20 s to an alternating magnetic field with a frequency of 1 MHz and a field strength of 100 Oe. 1 ml sterile LB medium was added, after which the sample was incubated for 45 min at 37° C. 10 μl of the sample was then spread on agar plates (LB medium, 75 μg/ml ampicillin, 50 μg/ml and 25 μg/ml X-gal). The plates were incubated at 37° C. overnight, whereupon the transfection frequency 2.5×10⁷ colonies/mg DNA was measured by counting the number of blue colonies/agar plate.

### EXAMPLE IV

### Transfection of E. coli LB121 with Plasmid-DNA pUC18 with Different Magnetically Inducible Particles Comparison Between Different Difunctional Molecules

20 μl of different suspensions of particles according to the present invention, where the divalent component in each suspension consists of antibody directed to OmpA, concanavalin A, amino groups and carboxyl groups and where the concentration of the magnetically inducible component characterised the suspension with a magnetic relative permeability of 1.002, was added to 0.5 μg pUC18 plasmid-DNA. The sample was incubated for 20 min and after that 10 μl cells was added, grown to a cell density of OD(600 nm)=0.4 concentrated 10 times and resuspended 0.1 M PBS buffer containing 0.15 M NaCl. After incubation for 30 min, the sample was exposed for 20 s to an alternating magnetic field with a frequency of 1 MHz and a field strength of 100 Oe. 1 ml sterile LB medium was added, whereupon the sample was incubated for 45 min at 37° C. 10 μl of the sample was spread on agar plates (LB medium, 75 μg/ml ampicillin, 50 μg/ml and 25 μg/ml X-gal). The plates were incubated at 37° C. overnight.

The different suspensions gave the following results:

| | Number of positive colonies | |
|---|---|---|
| Ferrofluid | per agar plate | |
| Reference (sample without ferrofluid) | 1 | 1 |
| ff-NH₃⁺ | 6 | |
| ff-COO⁻ | 3 | |
| ff-AntiOmpA | 20 | |
| ff-ConcanavalinA | 97 | |

1. A particle for use in transport of substances through biological membranes, wherein said particle contains at least one magnetically inducible material and at least one difunctional molecule with at least one binding site for said substance and with at least one binding site for said biological membrane.

2. A particle as claimed in claim 1, wherein said magnetically inducible material contains iron oxide, iron oxide hydrate, gamma Fe₂O₃, Fe₃O₄, iron oxides containing metal ions such as Co, Ni, Mn, Be, Mg, Ca, Ba, Sr, Cu, Zn, Pt, Al, Cr, Bi, rare earth metals or mixtures thereof.

3. A particle as claimed in claim 1, wherein said difunctional molecule is a lectin such as Concanavalin A, transferrin, avidin, selectins, DNA, RNA, antibiotics, hormones, polyelectrolytes, antibodies, antigen, synthetic peptide, peptide, virus protein, polylysin, DNA polymerase, RNA polymerase, ligase, exonucleases, endonucleases, zinc fingers, repressors or promoters.

4. A particle as claimed in claim 1, wherein said difunctional molecule is a recombinant fusion protein or a fusion molecule between at least two of the following units: lectin, such as Concanavalin A, transferrin, avidin, selectins, DNA, RNA, antibiotics, hormones, polyelectrolytes, antibodies, antigen, synthetic peptide, peptide, virus protein, polylysin, DNA polymerase, RNA polymerase, ligase, exonucleases, endonucleases, zinc fingers, repressors or promoters.

5. A particle as claimed in claim 1, wherein said particle is a particle with an average diameter in the range of about 1 nm to about 10 μm.

6. A particle as claimed in claim 1, wherein said particle contains an indicator such as a colourant, a fluorescent material, a radioactive material, a chemoluminescent material, or an enzyme.

7. A particle as claimed in claim 1, wherein said particle contains a bilayer membrane component which can be made up of, for instance, phospholipids and/or cholesterol.

8. A method for transporting substances through biological membranes, where the particle as claimed in claim 1 is mixed with membrane-enclosed structures and is allowed to be incubated for about 1 min to about 3 h, after which the formed particle membrane complex is exposed to an alternating magnetic field.

9. A method as claimed in claim 8, wherein the frequency of the alternating magnetic field is within the range of about 10 Hz to about 100 MHz with a field strength within the range of about 1 to about 100 Oerstedt.

10. A method for transporting substances according to claim 1 wherein said substance is selected from the group consisting of DNA, RNA, PNA, protein or part thereof, peptide, viruses, polymers, pharmaceutical preparations and steroids.

11. A method for biochemical work, transfection, transformation, gene transfer, gene expression control, cell differentiation control, protein expression control, protein synthesis, in vivo protein activity measurement, gene modification of vi ruses/protozoa/mould/bacteria and/or organelles therein/bacteriophages/plant cells and/or organelles therein/mammal cells/primary cells/stem cells wherein said particle of claim 1 is utilized.

12. A particle as claimed in claim 2, wherein said difunctional molecule is a lectin such as Concanavalin A, transferrin, avidin, selectins, DNA, RNA, antibiotics, hormones, polyelectrolytes, antibodies, antigen, synthetic peptide, peptide, virus protein, polylysin, DNA polymerase, RNA polymerase, ligase, exonucleases, endonucleases, zinc fingers, repressors or promoters.

13. A particle as claimed in claim 2, wherein said difunctional molecule is a recombinant fusion protein or a fusion molecule between at least two of the following units: lectin, such as Concanavalin A, transferrin, avidin, selectins, DNA, RNA, antibiotics, hormones, polyelectrolytes, antibodies, antigen, synthetic peptide, peptide, virus protein, polylysin, DNA polymerase, RNA polymerase, ligase, exonucleases, endonucleases, zinc fingers, repressors or promoters.

14. A particle as claimed in claim 3, wherein said difunctional molecule is a recombinant fusion protein or a fusion molecule between at least two of the following units: lectin, such as Concanavalin A, transferrin, avidin, selectins, DNA, RNA, antibiotics, hormones, polyelectrolytes, antibodies, antigen, synthetic peptide, peptide, virus protein, polylysin, DNA polymerase, RNA polymerase, ligase, exonucleases, endonucleases, zinc fingers, repressors or promoters.

15. A particle as claimed in claim 12, wherein said difunctional molecule is a recombinant fusion protein or a fusion molecule between at least two of the following units: lectin, such as Concanavalin A, transferrin, avidin, selectins, DNA, RNA, antibiotics, hormones, polyelectrolytes, antibodies, antigen, synthetic peptide, peptide, virus protein, polylysin, DNA polymerase, RNA polymerase, ligase, exonucleases, endonucleases, zinc fingers, repressors or promoters.

16. A particle as claimed in claim 2, wherein said particle is a particle with an average diameter in the range of about 1 nm to about 10 μm.

17. A particle as claimed in claim 2, wherein said particle contains an indicator such as a colourant, a fluorescent material, a radioactive material, a chemoluminescent material, or an enzyme.

18. A particle as claimed in claim 2, wherein said particle contains a bilayer membrane component which can be made up of, for instance, phospholipids and/or cholesterol.

19. A method for transporting substances through biological membranes, where the particle as claimed in claim 2 is mixed with membrane-enclosed structures and is allowed to be incubated for about 1 min to about 3 h, after which the formed particle membrane complex is exposed to an alternating magnetic field.

20. A method for transporting substances according to claim 2 wherein said substance is selected from the group consisting of DNA, RNA, PNA, protein or part thereof, peptide, viruses, polymers, pharmaceutical preparations and steroids.
